(19) United States
(12) Patent Application Publication
(10) Pub. No.: US 2020/0152308 A1
(43) Pub. Date: May 14, 2020

(54) MEDICATION-TAKING MANAGEMENT SYSTEM, MEDICATION-TAKING MANAGEMENT METHOD, MANAGEMENT DEVICE, AND PROGRAM STORAGE MEDIUM

(71) Applicant: NEC Corporation, Tokyo (JP)

(72) Inventors: Yuji OHNO, Tokyo (JP); Masahiro KUBO, Tokyo (JP)

(73) Assignee: NEC Corporation, Tokyo (JP)

(21) Appl. No.: 16/625,939

(22) PCT Filed: Jun. 18, 2018
(86) PCT No.: PCT/JP2018/023058

§ 371 Date: Dec. 23, 2019

(30) Foreign Application Priority Data
Jun. 28, 2017 (JP) 2017-126227

(51) Int. Cl. G16H 20/13; G01N 21/85; A61J 7/04; G06T 1/00; G06T 7/00

(52) U.S. Cl. CPC G16H 20/13; G01N 21/85; G06T 7/0002; G06T 1/0007; A61J 7/04

(57) ABSTRACT

In order to accurately manage medication-taking, a medication-taking management system is provided with: an irradiation unit; an image capture unit; an identification unit; and an output control unit. The irradiation unit irradiates a tablet that moves in a housing with light. The image capture unit captures an image of the tablet being irradiated. The identification unit, on the basis of information representing a feature of a tablet surface obtained from a captured image obtained by means of the image capture unit, identifies the tablet of which an image has been captured. The output control unit outputs an image capture time, indicating the time of acquisition of the captured image, in association with the result of identification.

### TECHNICAL FIELD

The present disclosure relates to a technique for managing medication-taking.

### BACKGROUND ART

There is a problem that a patient does not take a medication (non-compliance of a patient) without following a medication-taking instruction from a doctor, a pharmacist, and the like, which results in a waste of medical expenses. Specifically, there are problems that a medication prescribed for a patient is not taken and is discarded, resulting in an increase in cost, and a patient does not take a medication without following a medication-taking instruction, resulting in a decrease in a therapeutic effect, and the patient needs to consult a doctor again.

Further, a pharmaceutical company points out a possibility that it is unclear that a medication correctly exhibits an efficacy to a patient and a possibility that a side effect occurs in a patient, due to no medication-taking without following a medication-taking instruction. Further, a pharmaceutical company also points out that a medication to be originally consumed is not consumed, and as a result, a medication is not continuously distributed, resulting in a loss of a business opportunity.

Further, with progress of medical technologies in recent years, individualized medical care in which a therapeutic effect of a medication is maximized, such as preparation of a medication-taking plan conforming to a symptom and a constitution of an individual patient, has been started. Therefore, more correct medication-taking management is required.

When medication-taking management is performed, a method of recognizing a medication and managing both the recognized medication and a time is conceivable. As an example of a method of recognizing a medication, PTL 1 describes a method of recognizing a stamped character printed on a medication.

Further, PTL2 describes a method of improving color identification accuracy of a tablet when detecting an appearance defect in a production process of a press through package (PTP) sheet.

### CITATION LIST

### Patent Literature

[PTL 1] International Publication No. WO 2015/046044

[PTL 2] Japanese Unexamined Patent Application Publication No. 2006-194801

### SUMMARY OF INVENTION

### Technical Problem

For example, in a case of a tablet having two faces, a stamped character may be stamped only on one face. Therefore, in the technique described in PTL 1, there is a possibility that a stamped character is not recognized, depending on an image capture direction of the tablet. Therefore, when the technique described in PTL 1 is employed, medication-taking management may not be correctly performed. Further, the technique described in PTL 2 recognizes a medication by using prescription information, and therefore, when a medication taken by a user is not included in the prescription information, management about what medication the user takes may not be performed.

The present disclosure has been made in view of the above-described problems and a main object thereof is to accurately manage medication-taking.

### Solution to Problem

A medication-taking management system according to an example aspect of the present disclosure includes irradiation unit configured to irradiate a tablet moving in a housing with light, image capture unit configured to capture an image of the irradiated tablet, identification unit configured to identify the captured tablet, based on information representing a feature of a tablet surface acquired from a captured image acquired by the image capture unit, and output control unit configured to output an image capture time indicating a time of capturing the captured image and an identification result associated with the image capture time.

A medication-taking management method according to an example aspect of the present disclosure includes irradiating a tablet moving in a housing with light, capturing an image of the irradiated tablet, identifying the captured tablet, based on a feature of a tablet surface acquired from a captured image, and outputting an image capture time indicating a time of capturing the captured image and an identification result associated with the image capture time.

A management device according to an example aspect of the present disclosure includes identification unit configured to identify a tablet subjected to image capturing, based on a feature of a tablet surface acquired from a captured image acquired by capturing an image of a tablet irradiated with light, and output control unit configured to output an image capture time indicating a time of capturing the captured image and an identification result associated with the image capture time.

Note that, a computer program that achieves the above-described devices or method by using a computer, and a computer-readable non-transitory recording medium storing the computer program are also included in the scope of the present disclosure.

### Advantageous Effects of Invention

According to the present disclosure, medication-taking can be accurately managed.

### BRIEF DESCRIPTION OF DRAWINGS

FIG. 1 is a perspective view illustrating one example of a tablet removal case according to a first example embodiment.

FIG. 2 is an A-A-line arrow view of FIG. 1.

FIG. 3 is a B-B-line arrow view of FIG. 1.

FIG. 4 is a block diagram illustrating one example of a configuration in a tablet removal management system according to the first example embodiment.

FIG. 5 is a diagram illustrating one example of tablet information stored in a recording unit according to the first example embodiment.

FIG. 6 is a block diagram illustrating one example of a hardware configuration of a tablet-image capture device according to the first example embodiment.

FIG. 7 is a block diagram illustrating one example of a hardware configuration of a management device according to the first example embodiment.

FIG. 8 is a diagram illustrating one example of an area surrounded by a dashed-dotted line in FIG. 2.

FIG. 9 is a diagram illustrating one example of a captured image.

FIG. 10 is a diagram illustrating one example of tablet removal information.

FIG. 11 is a flowchart illustrating one example of a flow of processing of the tablet removal management system according to the first example embodiment.

FIG. 12 is a diagram illustrating one example of a structure of a tablet removal case according to a second example embodiment.

FIG. 13 is a block diagram illustrating one example of a configuration of a tablet removal management system according to the second example embodiment.

FIG. 14 is a diagram illustrating one example of tablet information stored in a recording unit according to the second example embodiment.

FIG. 15 is a diagram illustrating one example of tablet removal information.

FIG. 16 is a block diagram illustrating one example of a configuration of a tablet removal management system according to a third example embodiment.

### EXAMPLE EMBODIMENT

### First Example Embodiment

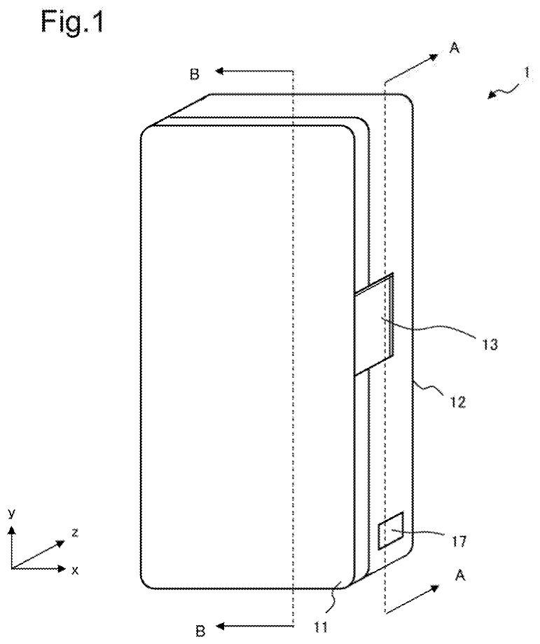

A first example embodiment of the present disclosure is described with reference to drawings. First, with reference to FIGS. 1 to 3, a physical structure of a tablet removal case (housing) 1 according to the present example embodiment is described. FIG. 1 is a perspective view illustrating one example of the tablet removal case 1 according to the present example embodiment. The tablet removal case 1 includes a lid unit 11 and a main body unit 12. A shape and a configuration of the tablet removal case 1 to be described according to the present example embodiment represent one example.

It is assumed that the tablet removal case 1 is substantially box-shaped, but the shape of the tablet removal case 1 is not specifically limited. The lid unit 11 is attached to the main body unit 12 via a hinge (not illustrated). Further, the lid unit 11 may include a latching unit 13 engaging with the main body unit 12.

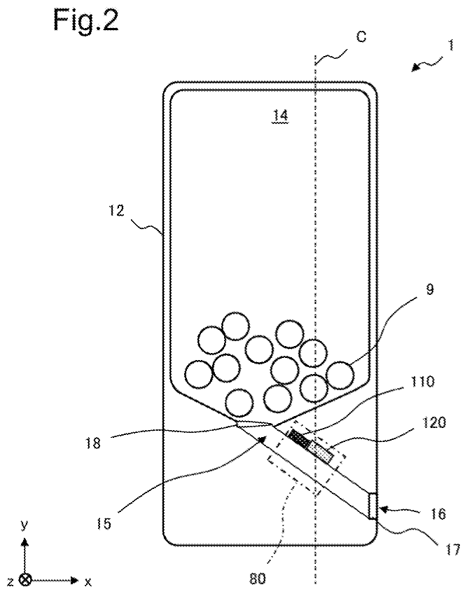

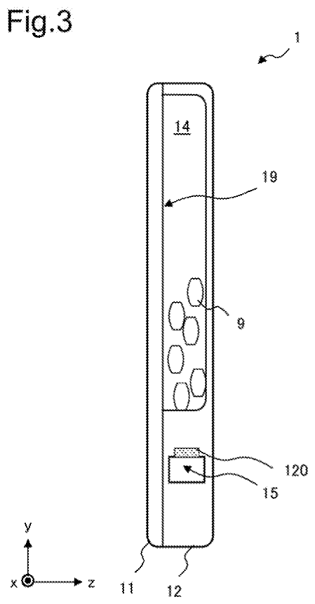

FIG. 2 is an A-A-line arrow view of FIG. 1. Further, FIG. 3 is a B-B-line arrow view of FIG. 1. In FIG. 2, a line segment C of a dashed line indicates a position of a B-B line in FIG. 1.

The main body unit 12 includes an accommodation unit 14 that accommodates a tablet 9. Note that, a material accommodated in the accommodation unit 14 is not limited to a tablet 9 and may be a medication (formulation) such as an encapsulated formulation and the like. When the lid unit 11 is opened, the main body unit 12 includes an opening 19 for inserting a tablet 9 into the accommodation unit 14. The opening 19 may have a size into which a tablet 9 can be inserted.

In the main body unit 12, a guide groove 15 that guides a tablet 9 to an outside of the tablet removal case 1 is extended from the accommodation 14. In other words, a space of the accommodation unit 14 and a space of the guide groove 15 form a coupled space. Further, the main body unit 12 includes an outlet 16 for taking out, to an outside of the tablet removal case 1, a tablet 9 accommodated in the accommodation unit 14 that is an inside of the tablet removal case 1. The outlet 16 includes an opening/closing lid 17, and when the opening/closing lid 17 is opened, a tablet 9 is removed from the outlet 16 to an outside of the tablet removal case 1.

A shape of the guide groove 15 is not specifically limited and may be a shape capable of guiding a tablet 9 from the accommodation unit 14 to the outlet 16. Further, a size of the accommodation unit 14 is not specifically limited and may be a size capable of accommodating a plurality of tablets 9.

The guide grove 15 includes an irradiation unit 110 that irradiates a tablet 9 with light and an image capture unit 120 that captures an image of a tablet 9 irradiated with light. The irradiation unit 110 and the image capture unit 120 are described in detail by changing the drawings.

The main body unit 12 includes a mechanism 18 that separates a space of the accommodation unit 14 and a space of the guide groove 15. The mechanism 18 may have a structure where a tablet 9 moving from the accommodation unit 14 to the guide groove 15 does not return from the guide groove 15 to the accommodation unit 14. The mechanism 18 may be a valve, for example, as illustrated in FIG. 2. A tablet 9 can be moved from the accommodation unit 14 to the guide groove 15 by moving the mechanism 18 in a direction of the guide groove 15.

Note that, between the lid unit 11 and the main body unit 12, a face capable of placing a wrapping body including a tablet 9 being one or more wrapped materials may be provided. In this case, the lid unit 11 may have an opening where a wrapping body is exposed. It may be possible that a wrapping body is sandwiched by the lid unit 11 having an opening and a face provided in the main body unit 12 and the wrapping body is accommodated in the tablet removal case 1.

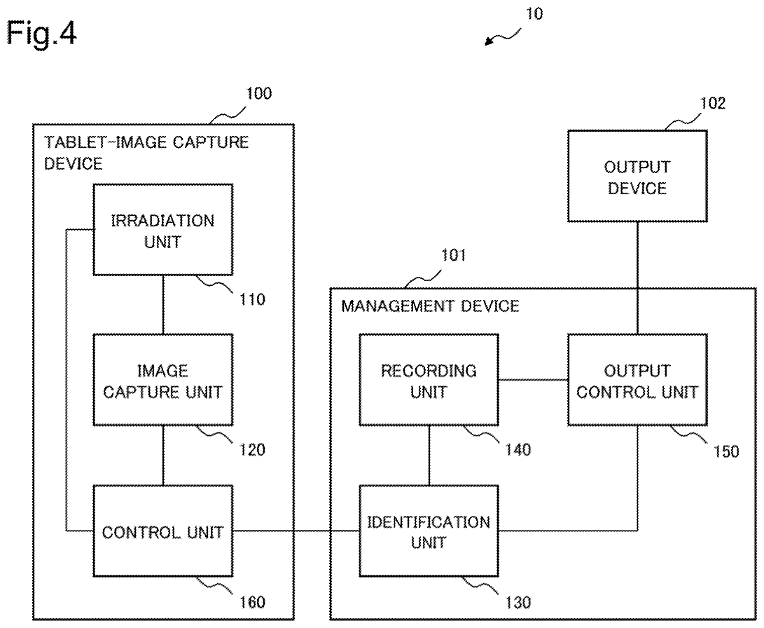

Next, with reference to FIG. 4, a configuration of a tablet removal management system (medication-taking management system) 10 according to the present example embodiment is described. FIG. 4 is a block diagram illustrating one example of a configuration of the tablet removal management system 10 according to the present example embodiment. As illustrated in FIG. 4, the tablet removal management system 10 includes a tablet-image capture device 100, a management device 101, and an output device 102. The tablet-image capture device 100 and the management device 101 are communicably connected to each other via a wired or wireless communication network. Further, the management device 101 and the output device 102 are communicably connected to each other via a wired or wireless communication network. Note that, according to the present example embodiment, while the tablet-image capture device 100, the management device 101, and the output device 102 each are described as a separate component, these may be integrally formed. Further, the tablet-image capture device 100 and the management device 101 may be integrally configured, and the management device 101 and the output device 102 may be integrally configured.

The tablet-image capture device 100 captures an image of a tablet that is passed through the guide groove 15 from the main body unit 12 of the tablet removal case 1 and is removed to an outside of the tablet removal case 1. The tablet-image capture device 100 includes an irradiation unit 110, an image capture unit 120, and a control unit 160.

The irradiation unit 110 irradiates a tablet 9 with light. Specifically, the tablet-image capture device 100 irradiates a tablet 9 moving in the guide groove 15 with light.

The image capture unit 120 captures an image of a tablet 9 irradiated with light. The image capture unit 120 supplies a captured image acquired by capturing an image of a tablet 9 to the management device 101 via the control unit 160. The image capture unit 120 may store a captured image in a local storage unit or a storage unit being not illustrated.

The control unit 160 controls the entirety of the tablet-image capture device 100. The control unit 160 may control a start or termination of light irradiation performed by the irradiation unit 110. Further, the control unit 160 may transmit an image capture instruction to the image capture unit 120. Thereby, the image capture unit 120 captures an image of a tablet 9. The control unit 160 may transmit, when detecting that, for example, a tablet 9 passes through the mechanism 18, an image capture instruction to the image capture unit 120. Further, the control unit 160 may control, when detecting that a tablet 9 passes through the mechanism 18, the irradiation unit 110 in such a way as to perform light irradiation.

Note that, control executed by the control unit 160 is not limited thereto. The control unit 160 may detect that, for example, a user grasps the tablet removal case 1 or the tablet removal case 1 is moved and thereby control the irradiation unit 110 and the image capture unit 120.

The control unit 160 supplies a captured image captured by the image capture unit 120 to the management device 101. Note that a captured image is associated with an image capture time indicating a time of acquiring the captured image by the image capture unit 120.

The management device 101 receives a captured image from the tablet-image capture device 100 and identifies a tablet 9 included in the captured image. The management device 101 identifies a type of a tablet 9. A type of a tablet 9 is classified based on a name of a tablet 9, a code identifying a tablet 9, or the like. According to the present example embodiment, the management device 101 is described as identifying a product name representing a name of a tablet 9. The management device 101 includes an identification unit 130, a recording unit 140, and an output control unit 150.

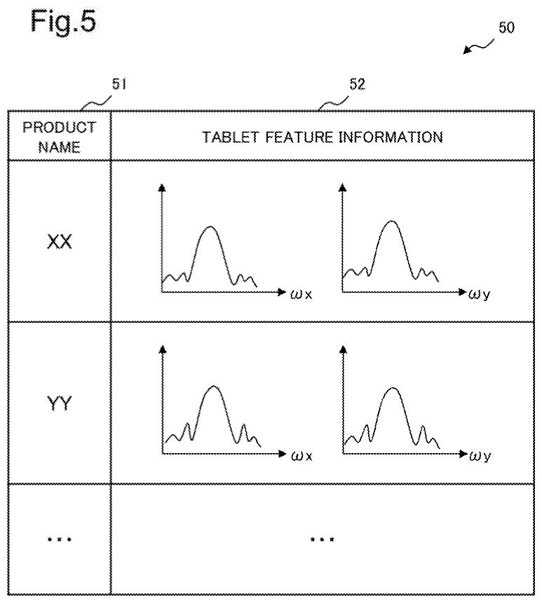

The recording unit 140 stores information (tablet information) relating to a tablet 9. Specifically, the recording unit 140 stores, as tablet information, information associating information representing a tablet 9 with information capable of identifying the tablet 9. FIG. 5 is a diagram illustrating one example of tablet information stored in the recording unit 140 according to the present example embodiment. Tablet information 50 includes a product name 51 and tablet feature information 52. A product name 51 is information representing a tablet 9. Note that information representing a tablet 9 is not limited to a product name 51 and may be an identification code for identifying a tablet 9.

Tablet feature information 52 is information capable of identifying that a tablet 9 is a tablet having what product name 51. Tablet feature information 52 may be information in a spatial frequency domain acquired, for example, by using, as learning data, a captured image acquired by capturing an image of a tablet 9 and by performing, for example, two-dimensional Fourier transform for a captured image being the learning data. In FIG. 5, by using learning data including a plurality of captured images, the learning data are subjected to two-dimensional Fourier transform and an average value of transformed values is designated as tablet feature information 52. Note that, FIG. 5 illustrates that a frequency of an x-axis component and a frequency of a y-axis component of a captured image each are allocated to a horizontal axis and a vertical axis is indicated as an intensity component, but tablet feature information 52 is not limited thereto. Tablet feature information 52 may be information acquired from an image previously provided or may be another piece of information.

The recording unit 140 may further stores, as tablet removal information, an identification result based on the identification unit 130. Tablet removal information is described later by changing the drawing.

The identification unit 130 identifies, from information representing a feature of a tablet surface acquired from a captured image supplied from the tablet-image capture device 100, a tablet 9 included in the captured image. Specifically, the identification unit 130 acquires information representing a feature of a tablet surface capable of identifying a tablet 9 included in a captured image from an image of an area of a tablet 9 in the captured image. The identification unit 130 acquires, for example, information representing a mottle of brightness from an image of an area of a tablet 9 in a captured image. Herein, a mottle of brightness is a change of brightness appearing due to irregularities and the like formed on a surface of a tablet 9 and is different from a pattern previously applied to a tablet 9. The identification unit 130 acquires information representing a mottle of brightness, for example, by converting a captured image to a spatial frequency domain. Note that, a type of information, acquired by the identification method 130, representing a feature of a tablet surface capable of identifying a tablet 9 included in a captured image is not specifically limited and may be information of the same type as information stored in the recording unit 140. Herein, the recording unit 140 stores, as information representing a feature of a tablet surface capable of identifying a tablet 9, information in a spatial frequency domain expressing an image of a surface of a tablet 9, and therefore the identification unit 130 acquires, from a captured image, a value in which the captured image is converted to a spatial frequency domain.

The identification unit 130 compares information acquired from a captured image with tablet feature information 52 stored in the recording unit 140 and specifies a product name 51 associated with tablet feature information 52 which are most similar to the information acquired from the captured image. Note that, a method of specifying a tablet 9 by using the identification unit 130 is not limited thereto, and the identification unit 130 may execute the comparison, for example, based on a predetermined condition. When, for example, information capable of identifying a tablet 9 is information in a spatial frequency domain, pieces of information in a frequency band where a frequency component is equal to or larger than a predetermined value may be compared. In this manner, an identification method executed by the identification unit 130 is not specifically limited. The identification unit 130 supplies, as an identification result, an identified product name 51 to the output control unit 150.

The output control unit 150 outputs a product name 51 being an identification result and an image capture time of a captured image in association with each other. Specifically, the output control unit 150 generates a control signal for controlling associated information in such a way as to be output to the output device 102 and outputs the control signal to the output device 102. The output control unit 150 outputs, to the output device 102, a control signal for controlling the output device 102 in such a way as to output associated information, in a form according to the output device 102. The output control unit 150 may store associated information in the recording unit 140 as tablet removal information.

The output device 102 executes output based on a control signal from the management device 101. The output device 102 may be, for example, a display device such as a display and the like or may be a terminal device including a display. Further, the output device 102 is not limited thereto and may be a printer or a device that file-outputs information included in a received signal. Further, the output device 102 may be a speaker that performs voice output and the like. The output device 102 may be a robot-type user interface or a wearable device including one or a plurality of output functions such as a display, a speaker, a combination thereof, and the like.

When the output device 102 is, for example, a display device such as a display (display unit) or a terminal device including a display, the output control unit 150 outputs a control signal for screen-displaying an identification result and an image capture time to the output device 102. Thereby, the output device 102 displays when and what tablet 9 is taken on a screen. Therefore, the tablet removal management system 10 can cause a manager and the like operating the output device 102 to gain an understanding of this status.

Further, when the output device 102 is a device that file-outputs received information, the output control unit 150 outputs, to the output device 102, a control signal for file-outputting information representing an identification result. Thereby, the output device 102 can output, as a file, information indicating when and what tablet 9 is taken. Therefore, the tablet removal management system 10 stores such a file and thereby can correctly manage a medication-taking status of a user.

Further, when the output device 102 is a speaker that performs voice output, the output control unit 150 may output, to the output device 102, a control signal for outputting a voice representing an identification result. A control signal generated by the output control unit 150 is, for example, when, an image capture time associated with an identification result is different from a previously set time, a signal for causing the output device 102 to output a warning sound indicating that a tablet 9 is not removed at the set time. Further, a control signal generated by the output control unit 150 is, for example, a signal for causing the output device 102 to output a warning sound indicating that a predetermined number or more of tablets 9 are removed within a predetermined time. Thereby, a manager using the tablet removal management system 10 can understand, by hearing the warning sound, that medication-taking of a tablet 9 is not performed without following a previously determined criterion.

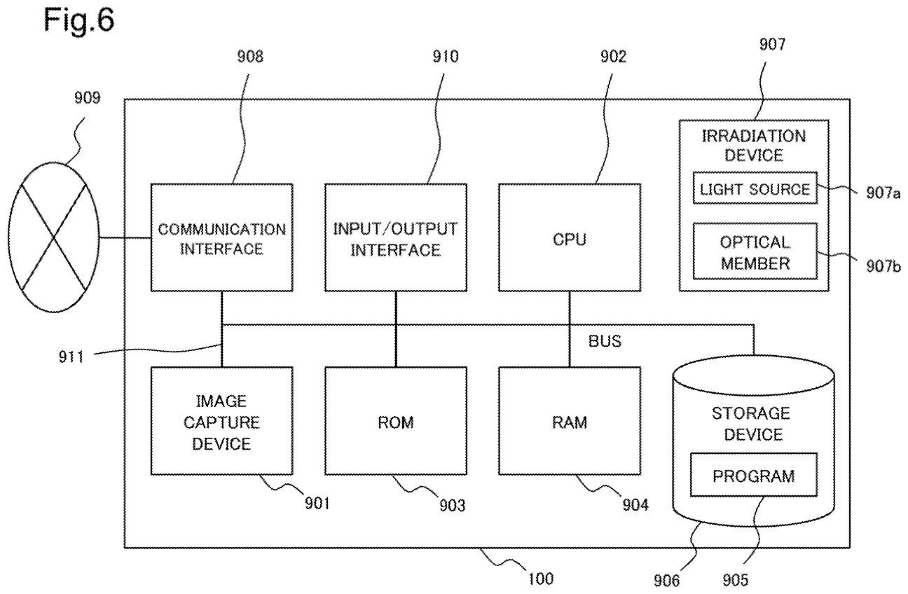

Next, a hardware configuration of each of the tablet-image capture device 100 and the management device 101 of the tablet removal management system 10 is described. FIG. 6 is a block diagram illustrating one example of a hardware configuration of the tablet-image capture device 100 of the tablet removal management system 10 according to the present example embodiment. The tablet-image capture device 100 includes the following configuration as one example.

Image capture device 901

Central processing unit (CPU) 902

Read only memory (ROM) 903

Random access memory (RAM) 904

Program 905 loaded on the RAM 904

Storage device 906 storing the program 905

Irradiation device 907 including a light source 907a and an optical member 907b

Input/output interface 910 inputting/outputting data

Bus 911 connecting components

Further, the tablet-image capture device 100 may include, as illustrated in FIG. 6, a communication interface 908 for connection to a communication network 909.

The irradiation unit 110 is achieved by the irradiation device 907. The light source 907a is, for example, a laser or a light emitting diode (LED). The optical member 907b is, for example, a beam expander, a collimator lens, or a combination thereof. The optical member 907b may be appropriately selected according to the light source 907a. According to the present example embodiment, light emitted from the light source 907a is collimated by the optical member 907b. Thereby, the irradiation device 907 emits collimated light or substantially parallel light.

The image capture unit 120 is achieved by the image capture device 901 such as a camera including an imaging element such as a charge-coupled device (CCD) image sensor or a complementary metal-oxide-semiconductor (CMOS) image sensor, and a lens.

The control unit 160 is achieved, for example, by acquiring and executing, by using the CPU 902, the program 905 achieving a function of the control unit 160. The program 905 achieving a function of the control unit 160 is, for example, previously stored, in the storage device 906 or on the ROM 903, and is loaded onto the RAM 904 and executed by the CPU 902, as necessary. Note that, the program 905 may be supplied to the CPU 902 via the communication network 909.

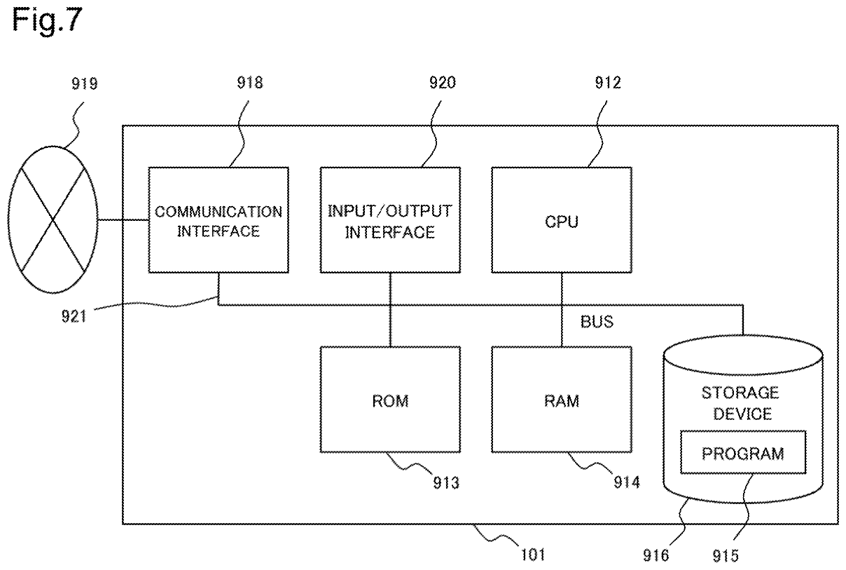

FIG. 7 is a block diagram illustrating one example of a hardware configuration of the management device 101 of the tablet removal management system 10 according to the present example embodiment. The management device 101 includes, as one example, the following configuration.

Central processing unit (CPU) 912

Read only memory (ROM) 913

Random access memory (RAM) 914

Program 915 loaded on the RAM 914

Storage device 916 storing the program 915

Input/output interface 920 inputting/outputting data

Bus 921 connecting components

The identification unit 130 and the output control unit 150 are achieved, for example, by acquiring and executing, by using the CPU 912, the program 915 achieving a function of each of the identification unit 130 and the output control unit 150. The program 915 achieving a function of each of the identification unit 130 and the output control unit 150 is previously stored, for example, in the storage device 916 or on the ROM 913, and is loaded onto the RAM 914, and executed by the CPU 912 as necessary. Note that the program 915 may be supplied to the CPU 912 via a communication network 919.

The recording unit 140 may be achieved, for example, by the storage device 916 or may be achieved by a storage device separate from the storage device 916. Further, the recording unit 140 may be achieved in a device different from the management device 101, instead of being included in the management device 101. In this case, the identification unit 130 may access the recording unit 140 via the communication network 919.

Note that, a part or all of components of the tablet-image capture device 100 and the management device 101 are achieved by another general-purpose or dedicated circuit, a processor or the like, or a combination thereof. These may be configured by a single chip or may be configured by a plurality of chips connected via a bus. Further, a part or all of components of the tablet-image capture device 100 and the management device 101 may be achieved by a combination of the above-described circuit and the like and a program. Further, the tablet-image capture device 100 and the management device 101 may include a drive device for executing reading/writing from/onto a storage medium.

Further, the tablet-image capture device 100 and the management device 101 each may include a component other than components illustrated in FIGS. 6 and 7. The tablet-image capture device 100 may include, for example, a battery and the like. The components illustrated in FIG. 6 are accommodated, for example, in the main body unit 12 of the tablet removal case 1. Further, the components illustrated in FIG. 7 are mounted, for example, on a server device including the management device 101.

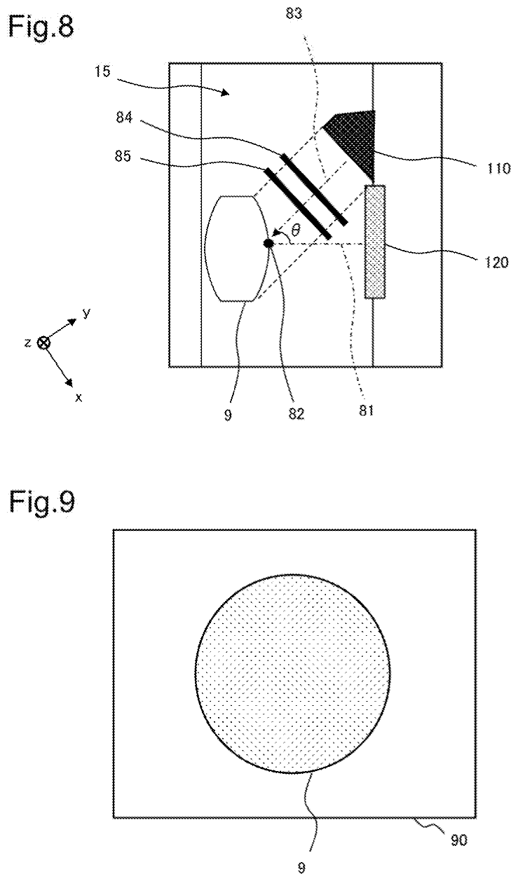

FIG. 8 is a diagram illustrating one example of an area 80 surrounded by a dashed-dotted line in FIG. 2. As illustrated in FIG. 8, the irradiation unit 110 and the image capture unit 120 are disposed on a path of the guide groove 15. The image capture unit 120 captures an image of a tablet 9 when the tablet 9 passing through the guide groove 15 is located within an angle of view of the image capture unit 120. Note that, the image capture unit 120 may capture an image of a tablet 9 passing through the guide groove 15 by continuously capturing an image of the guide groove 15, or may perform image capturing based on an image capture instruction transmitted from the control unit 160. When, for example, an image capture instruction including information indicating a time at which a tablet 9 is located within an angle of view of the image capture unit 120 is transmitted from the control unit 160, the image capture unit 120 may perform image capturing according to the image capture instruction, when the tablet 9 is located within the angle of view of the image capture unit 120. Note that the image capture unit 120 preferably captures an image of a tablet 9 at a position where the tablet 9 faces the image capture unit 120.

Further, the irradiation unit 110 is disposed in a vicinity of the image capture unit 120. Specifically, the irradiation unit 110 is disposed at a position where an angle (incident angle θ) formed by incident light 83 entering a position 82 at which an optical axis 81 of the image capture unit 120 and a tablet 9 intersect with each other and the optical axis 81 is equal to or larger than a predetermined angle. Note that, as described above, the irradiation unit 110 achieved by the irradiation device 907 preferably irradiates a tablet 9 with parallel light or substantially parallel light (light in which an area (irradiation area) of emitted light on any face (e.g. a first face 84) projected by light emitted from the light source 907a and an irradiation area on any another face (e.g. a second face 85) parallel to the any face preferably fall within a predetermined range). Thereby, compared with when the irradiation unit 110 emits diffused light (light in which at least one of an irradiation area on the first face 84 or an irradiation area on the second face 85 does not fall within a predetermined range), a mottle of illuminance in a captured image captured by the image capture unit 120 can be avoided.

Further, the tablet removal case 1 may include two or more irradiation units 110. The tablet removal case 1 may include, for example, a plurality of irradiation units 110 in a position where an incident angle θ is the same. Thereby, the image capture unit 120 can image a captured image having no shadow on a tablet 9.

FIG. 9 is a diagram illustrating one example of a captured image. When the image capture unit 120 captures an image of a tablet 9 located within an angle of view, a captured image 90 includes the tablet 9 as illustrated in FIG. 9. A mottle of brightness appears on a surface of the tablet 9 included in the captured image 90. Thereby, the identification unit 130 can identify the tablet 9 by using information able to be acquired from the mottle of brightness.

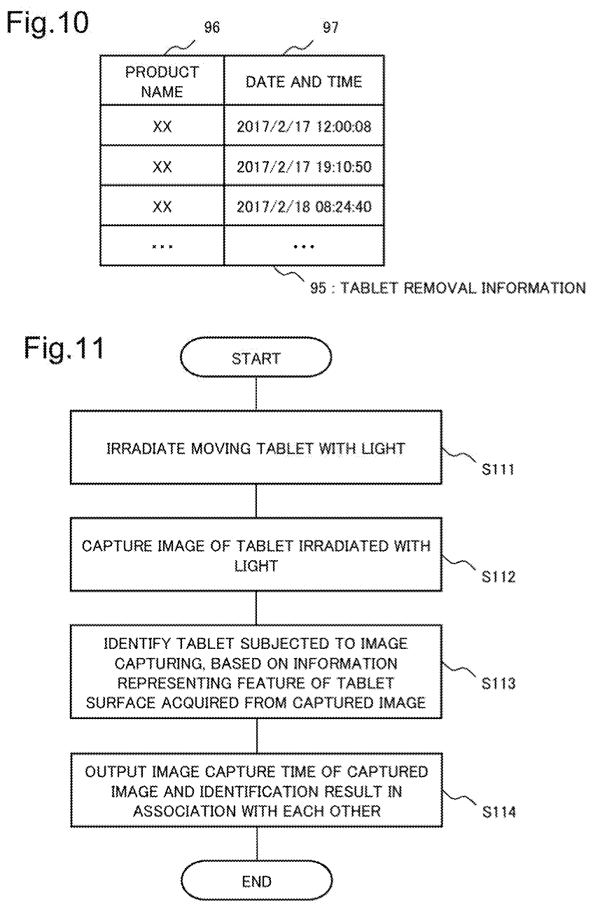

FIG. 10 is a diagram illustrating one example of tablet removal information 95 stored in the recording unit 140. Tablet removal information 95 includes a product name 96 and a date and time 97 as illustrated in FIG. 10. A product name 96 identifies a tablet 9, similarly to a product name 51. The output control unit 150 may store, in the recording unit 140, an image capture time of a captured image used when identifying a tablet 9 as a date and time 97 of tablet removal information 95 in association with a product name 96. Note that the output control unit 150 may control the tablet removal information 95 in such a way as to be output by the output device 102 as a file.

Thereby, the management device 101 can manage information representing that a tablet 9 is removed.

FIG. 11 is a flowchart illustrating one example of a flow of processing of the tablet removal management system 10 according to the present example embodiment. As illustrated in FIG. 11, the irradiation unit 110 irradiates a tablet 9 moving from the tablet removal case 1 to an outside with light (step S111). The image capture unit 120 captures an image of the tablet 9 irradiated with light (step S112).

The identification unit 130 identifies the tablet 9 subjected to image capturing, based on information representing a feature of a tablet surface acquired from the captured image (step S113). Then, the output control unit 150 outputs an image capture time of the captured image and an identification result in association with each other (step S114).

From the above, the tablet removal management system 10 terminates processing.

The tablet removal management system 10 according to the present example embodiment includes, as described above, the irradiation unit 110 that irradiates a tablet 9 moving from a tablet removal case 1 to an outside with light, the image capture unit 120 that captures an image of the tablet 9 irradiated with light, the identification unit 130 that identifies the tablet 9 subjected to image capturing, based on the captured image, and the output control unit 150 that outputs an image capture time of the captured image and an identification result in association with each other.

Thereby, the identification unit 130 identifies a tablet 9, based on a captured image of a tablet 9 irradiated with light, for example, based on information representing a change of brightness of the tablet 9. Thereby, the identification unit 130 can identify, even when, for example, a printed character printed on a tablet 9 is not included in a captured image, what tablet a tablet 9 removed from the tablet removal case 1 is. The output unit 150 outputs a result identified accurately in this manner together with an image capture time, and thereby from a result acquired from the output, the tablet removal management system 10 can accurately manage medication-taking of a user. Therefore, for example, a manager managing medication-taking of a user can correctly understand a status of medication-taking of a user.

Further, when the identification unit 130 identifies a tablet by using information of at least any one of a size, a shape, or a color of a tablet 9 or a character printed on the tablet 9, the tablet removal management system 10 can more accurately identify a tablet 9 removed from the tablet removal case 1.

### Second Example Embodiment

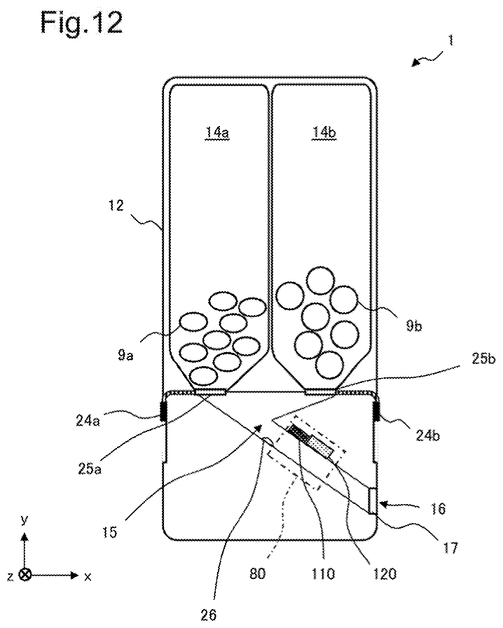

A second example embodiment of the present disclosure is described with reference to drawings. First, with reference to FIG. 12, a physical structure of a tablet removal case 1 according to the present example embodiment is described. FIG. 12 illustrates another example of the A-A line arrow view of FIG. 1. As illustrated in FIG. 12, a main body unit 12 of the tablet removal case 1 according to the present example embodiment includes a plurality of accommodation units 14a and 14b that each accommodate a tablet 9. According to the present example embodiment, description is made assuming that the accommodation unit 14a accommodates a tablet 9a and the accommodation unit 14b accommodates a tablet 9b different in product name from the tablet 9a.

The tablet removal case 1 includes slide buttons 24a and 24b that each receive input from a user. Further, the main body unit 12 includes a mechanism 25a that separates a space of the accommodation unit 14a and a space of a guide groove 15 and a mechanism 25b that separates a space of the accommodation unit 14b and a space of the guide groove 15. The mechanism 25a may have a structure where a tablet 9a moving from the accommodation unit 14a to the guide groove 15 does not return to the accommodation unit 14a from the guide groove 15. According to the present example embodiment, as illustrated in FIG. 12, it is assumed that the mechanism 25a is plate-shaped. The mechanism 25a is coupled with the slide button 24a. When a user slides the slide button 24a, for example, in a y-axis negative direction of FIG. 12, the mechanism 25a coupled with the slide button 24a moves, for example, in an x-axis negative direction of FIG. 12. Thereby, a partition between the accommodation unit 14a and the guide groove 15 is removed, and a tablet 9a in the accommodation unit 14a can move to the guide groove 15. A configuration of the slide button 24b and the mechanism 25a is similar to the configuration of the slide button 24a and the mechanism 25a.

Note that, according to the present example embodiment, when a tablet 9a and a tablet 9b are not distinguished or are collectively referred to, these are simply referred to as a tablet 9.

According to the present example embodiment, a configuration in which the tablet removal case 1 includes slide buttons 24a and 24b and mechanisms 25a and 25b is described, but similarly to the tablet removal case 1 according to the first example embodiment described above, a mechanism 18 that separates between spaces of the accommodation unit 14a and the accommodation unit 14b and a space of the guide groove 15 may be provided. Further, the tablet removal case 1 illustrated in FIG. 2 may include a slide button and a mechanism coupled with the slide button.

The guide groove 15 of the tablet removal case 1 according to the present example embodiment includes, similarly to the first example embodiment described above, an irradiation unit 110 and an image capture unit 120. Further, the guide groove 15 includes a mechanism 26 that temporarily stops passing of a tablet 9 in the guide groove 15. The mechanism 26 is disposed in a position inside the guide groove 15 closer to the accommodation unit 14a and 14b than the irradiation unit 110. Thereby, for example, an interval of a plurality of tablets 9 attempting to continuously pass through the guide groove 15 can be increased. Therefore, the image capture unit 120 can separately capture an image of each of a plurality of tablets 9. Note that, a shape of the mechanism 26 is not specifically limited and may be a convex shape as illustrated in FIG. 12 or another shape.

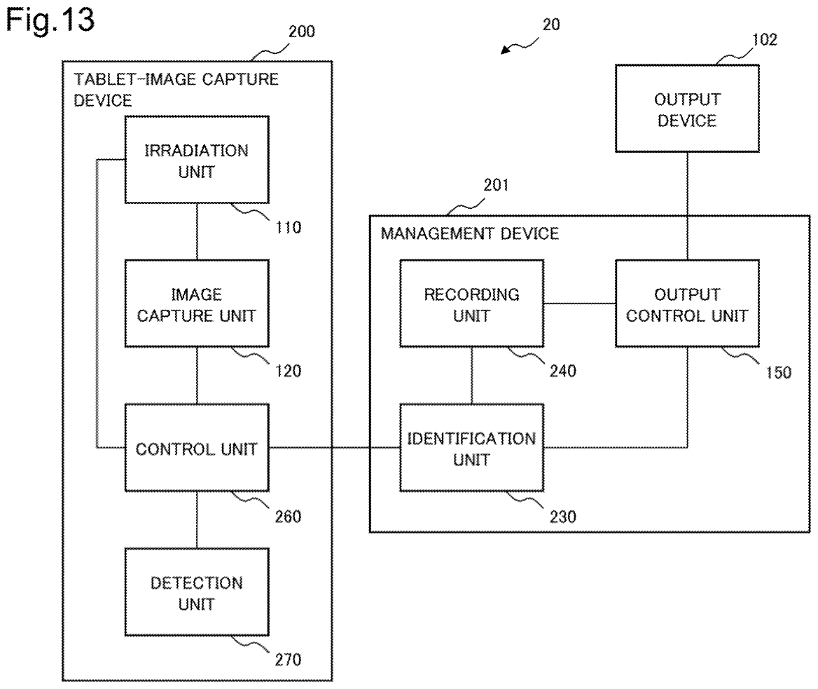

FIG. 13 is a diagram illustrating one example of a configuration of a tablet removal management system 20 according to the present example embodiment. The tablet removal management system 20 includes a tablet-image capture device 200, a management device 201, and an output device 102.

The tablet-image capture device 200 includes the irradiation unit 110, the image capture unit 120, the control unit 260, and a detection unit 270. The tablet-image capture device 200 includes a control unit 260 instead of the control unit 160 of the tablet-image capture device 100 and further includes the detection unit 270.

The detection unit 270 detects a signal based on input from an outside. A signal based on input from an outside is, for example, a signal indicating input of a user to the slide button 24 described above. When a user slides the slide button 24a, for example, in a y-axis negative direction illustrated in FIG. 12, the detection unit 270 detects a signal indicating that the slide button 24a is slid. In other words, the detection unit 270 detects a movement of the mechanism 25a for coupling a space of the guide groove 15 and a space of the accommodation unit 14a. Then, the detection unit 270 supplies a detection result to the control unit 260.

The control unit 260 controls the entirety of the tablet-image capture device 200, similarly to the control unit 160. Further, the control unit 260 may control one or both of the irradiation unit 110 and the image capture unit 120 according to a detection result based on the detection unit 270. When, for example, the detection unit 270 detects a signal indicating that the slide button 24a is slid, the control unit 260 may control a light source 907a in such a way as to be lighted, based on the detection result. Then, the control unit 260 may control the image capture unit 120 in such way as to perform image capturing after an elapse of a predetermined time from lighting of the light source 907a. Further, when the slide button 24a is slid, for example, in a y-axis positive direction and the detection unit 270 detects that a return is made to the state of FIG. 12, the control unit may turn off the light source 907a, based on the detection result.

Note that, the control unit 260 may control, when the light source 907a is lighted and thereafter a predetermined time elapses, the irradiation unit 110 in such a way as to turn off the light source 907a. In this manner, the control unit 260 controls one or both of the irradiation unit 110 and the image capture unit 120 according to a detection result of the detection unit 270, and thereby power consumption of one or both of the irradiation unit 110 and the image capture unit 120 can be reduced, compared with when there is no control by the control unit 260.

Note that, a signal based on input from an outside, detected by the detection unit 270, is not limited to a signal indicating that the slide button 24 is slid. The detection unit 270 may detect a change of the tablet removal case 1 due to pressure. When the tablet removal case 1 has, for example, a configuration in which a wrapping body can be fixed between a lid unit 11 and the main body unit 12, a pressure is applied to the tablet removal case 1 when a user pushes out a tablet 9 included in the wrapping body. Due to the pressure, the tablet removal case 1 bends by a predetermined amount. The detection unit 270 may detect the bending. In this case, the tablet removal case 1 may include a sensor for detecting bending and does not need to include a slide button 24 and a mechanism 25 coupled with the slide button 24. Therefore, when the detection unit 270 that detects bending of the tablet removal case 1 is provided, the tablet removal case 1 can be formed with a simple structure, compared with when the slide button 24 and the mechanism 25 are provided.

Further, the detection unit 270 may be achieved by a switch for detection depressed by a tablet 9. In other words, the tablet removal case 1 may include a switch for detection depressed by a tablet 9, instead of the slide button 24 and the mechanism 25. The switch may be disposed in a position depressed by a tablet 9 and may be disposed between a space of the accommodation unit 14 and the space of a guide groove 15.

Further, the detection unit 270 may detect a drop of a tablet 9. In other words, the tablet removal case 1 may include, as the detection unit 270, a sensor for detecting a drop of a tablet 9 inside the accommodation unit 14, instead of the slide button 24 and the mechanism 25. The sensor may be sheet-shaped or have another shape. The sensor may be achieved, for example, by a piezosensor or an acceleration sensor. The sensor may be disposed in a position where a drop of a tablet 9 can be detected, and the position is not specifically limited.

The management device 201 receives a captured image from the tablet-image capture device 200 and identifies a tablet 9 included in the captured image. The management device 201 identifies a type of a tablet 9, similarly to the management device 101. The management device 201 includes an identification unit 230, a recording unit 240, and an output control unit 150.

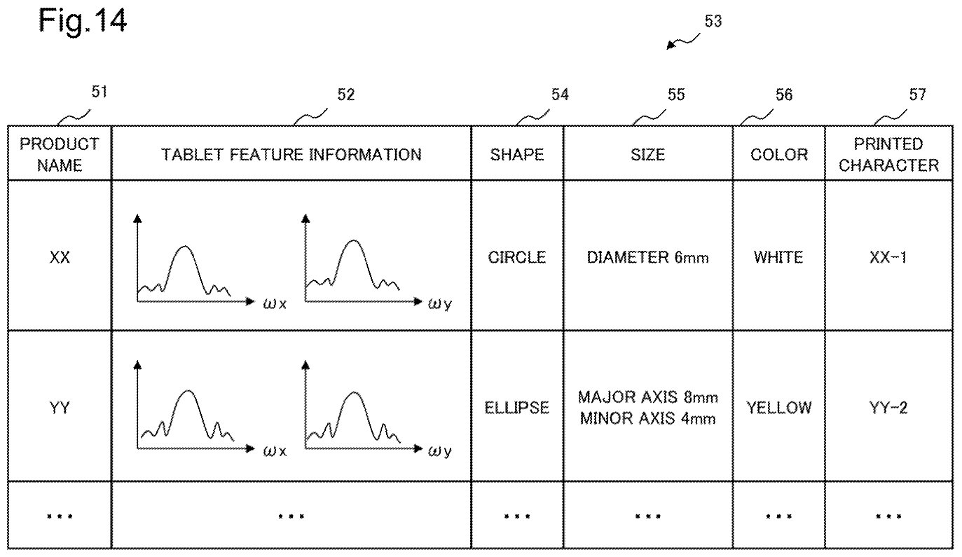

The recording unit 240 stores tablet information of a tablet 9. One example of tablet information according to the present example embodiment is illustrated in FIG. 14. Tablet information 53 includes a product name 51, tablet feature information 52, a shape 54 that is information indicating a shape of the tablet 9, a size 55 representing a size of the tablet 9, a color 56 representing a color of the tablet 9, and a printed character 57 representing a character printed on the tablet 9. A shape 54 is assumed to represent an approximate shape on a projection plane in which an area is maximum but may be any piece of information representing a shape of a tablet 9. A color 56 may be any one expressing a color and may be, for example, an RGB value.

Note that, the recording unit 240 may store tablet removal information, similarly to the recording unit 140 described above.

The identification unit 230 identifies, from a captured image supplied from the tablet-image capture device 100, a tablet 9 included in the captured image, similarly to the identification unit 130 described above. The identification unit 230 may acquire, from a captured image, at least any one of a shape, a size, a color, or a printed character of a tablet 9, in addition to information representing a mottle of brightness. Then, the identification unit 230 may refer to tablet information 53 stored in the recording unit 240, based on acquired information, and thereby identify a tablet 9 included in a captured image.

Thereby, the identification unit 230 can more accurately identify a tablet 9.

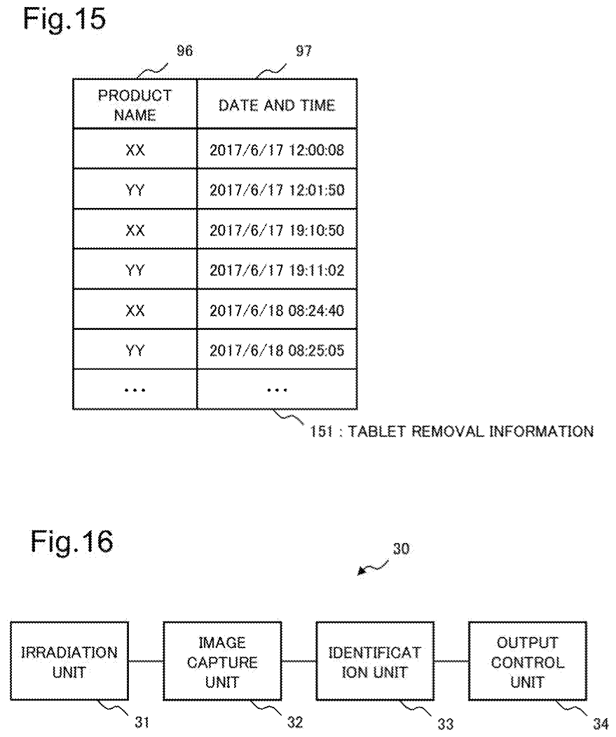

FIG. 15 is a diagram illustrating one example of tablet removal information 151 stored in the recording unit 240. Tablet removal information 151 includes a product name 96 and a date and time 97, similarly to tablet removal information 95. A product name 96 identifies a tablet 9, similarly to a product name 51. As illustrated in FIG. 12, even when a plurality of tablets 9 having different product names 51 are included in the tablet removal case 1, the identification unit 230 identifies a type of each removed tablet 9. Therefore, the output control unit 150 can output, as illustrated in FIG. 15, an image capture time of a tablet 9 with respect to each tablet 9. Therefore, the management device 201 can accurately manage medication-taking of a user of the tablet removal case 1.

Note that, according to the present example embodiment, the management device 201 may previously register, as tablet information 53, tablet feature information 52 of each of tablets 9 accommodated in the tablet removal case 1. In this case, the management device 201 may register, for each of tablets 9, not only a product name 51 but also information capable of individually identifying a tablet 9 (information for identifying an individual piece of a tablet 9). Then, the identification unit 230 may identify, during identification, an individual piece of each of tablets 9, by using information capable of individually identifying a tablet 9. Thereby, it is possible to more correctly manage whether a user takes a tablet 9 prescribed for the user.

Further, the recording unit 240 may store, as tablet information 53, information of a tablet 9 prescribed for a user with respect to each piece of information for identifying the tablet removal case 1 or each user using the tablet removal case 1. Thereby, when, for example, a tablet 9 that is not prescribed for a user is accommodated in the tablet removal case 1 and the tablet 9 is removed from the tablet removal case 1, the output control unit 150 can report that a tablet 9 removed by a doctor or a user is a tablet 9 that is not prescribed. Therefore, the management device 201 can manage that a user takes a non-prescribed tablet 9.

Further, the control unit 260 may acquire tablet information 53 from the recording unit 240 of the management device 201 and control the irradiation unit 110 in such a way as to modify an irradiation area of light emitted by the irradiation unit 110 according to a size 55. Further, the control unit 260 may control an angle of view of the image capture unit 120 in such a way as to be modified according to a size 55. When a captured image of a tablet 9 imaged via such control is used for identification, the identification unit 230 can enhance accuracy in identification of a tablet 9.

### Third Example Embodiment

Next, a third example embodiment of the present disclosure is described with reference to FIG. 16. According to the present example embodiment, a minimum configuration that solves the problem of the present disclosure is described.

FIG. 16 is a block diagram illustrating one example of a configuration of a tablet removal management system (medication-taking management system) 30 according to the present example embodiment. As illustrated in FIG. 16, the tablet removal management system 30 includes an irradiation unit 31, an image capture unit 32, an identification unit 33, and an output control unit 34.

The irradiation unit 31 includes a function of the irradiation unit 110. The irradiation unit 31 irradiates a tablet moving in a housing with light. Specifically, the irradiation unit 31 irradiates a tablet moving from a housing to an outside with light. The irradiation unit 31 is achieved, for example, by the irradiation unit 907 illustrated in FIG. 6.

The image capture unit 32 includes a function of the image capture unit 120. The image capture unit 32 captures an image of a tablet irradiated with light. The image capture unit 32 is achieved, for example, by the image capture device 901 illustrated in FIG. 6.

The identification unit 33 includes a function of the identification unit 130 or the identification unit 230. The identification unit 33 identifies a tablet subjected to image capturing, based on information representing a feature of a tablet surface acquired from a captured image acquired by the image capture unit 32.

The output control unit 34 includes a function of the output control unit 150. The output control unit 34 outputs an image capture time indicating a time of acquiring a captured image and an identification result in association with each other. The identification unit 33 and the output control unit 34 is achieved, for example, by acquiring and executing, by using the CPU 912, the program 915 illustrated in FIG. 7 achieving a function of each of the identification unit 33 and the output control unit 34.

In this manner, the identification unit 33 of the tablet removal management system 30 according to the present example embodiment identifies a tablet, based on information representing a feature of a tablet surface acquired from a captured image of the tablet irradiated with light, for example, based on information representing a change of brightness in an image of an area of a tablet in a captured image. Thereby, the identification unit 33 can identify, even when, for example, a printed character printed on a tablet is not included in a captured image, what tablet a tablet removed from a housing is. The output control unit 34 outputs a result identified accurately in this manner, together with an image capture time, and thereby from a result acquired by the output, the tablet removal management system 30 can accurately manage medication-taking of a user. Therefore, for example, a manager managing medication-taking of a user can correctly understand a status of medication-taking of a user.

Note that, units illustrated in FIG. 16 may be achieved by the same device. Further, similarly to the first and second example embodiments, the irradiation unit 31 and the image capture unit 32 may be mounted on a tablet-image capture device, and the identification unit 33 and the output control unit 34 may be mounted on a management device communicably connected to the tablet-image capture device. Further, a management device including the identification unit 33 and the output control unit 34 illustrated in FIG. 16 and a management method of medication-taking by the management device are also included in the scope of the present disclosure. At that time, the identification unit 33 may identify, based on a captured image acquired by capturing an image of a tablet irradiated with light, the tablet subjected to image capturing. Thereby, the management device including the identification unit 33 and the output control unit 34 can produce an advantageous effect similar to that of the tablet removal management system 30 described above.

Note that, example embodiments described above are preferred example embodiments of the present disclosure and the scope of the present disclosure is not limited to the example embodiments, and it is possible for those of ordinary skill in the art to make adjustments and substitutions of the example embodiments without departing from the gist of the present disclosure and construct forms subjected to various modifications.

The whole or part of the example embodiments disclosed above can be described as, but not limited to, the following supplementary notes.

(Supplementary Note 1)

A medication-taking management system comprising:

irradiation unit configured to irradiate a tablet moving in a housing with light;

image capture unit configured to capture an image of the irradiated tablet;

identification unit configured to identify the captured tablet, based on a feature of a tablet surface acquired from a captured image acquired by the image capture unit; and

output control unit configured to output an image capture time indicating a time of capturing the captured image and an identification result associated with the image capture time.

(Supplementary Note 2)

The medication-taking management system according to supplementary note 1, wherein

the identification unit identifies the tablet, based on a change of brightness in an area of the tablet in the captured image.

(Supplementary Note 3)

The medication-taking management system according to supplementary note 2, wherein

the identification unit identifies the tablet by using at least any one of a size, a shape, or a color of the tablet, or a character printed on the tablet.

(Supplementary Note 4)

The medication-taking management system according to any one of supplementary notes 1 to 3, wherein

the irradiation unit emits light where an irradiation area of light with respect to a first face and an irradiation area of light on a second face parallel to the first face fall within a predetermined range.

(Supplementary Note 5)

The medication-taking management system according to any one of supplementary notes 1 to 4, further comprising:

detection unit configured to detect a signal based on input from an outside; and

control unit configured to control at least one of the irradiation unit and the image capture unit according to a detection result by the detection unit.

(Supplementary Note 6)

The medication-taking management system according to supplementary note 5, wherein

the control unit controls at least one of the irradiation unit and the image capture unit according to information relating to the tablet.

(Supplementary Note 7)

The medication-taking management system according to any one of supplementary notes 1 to 6, wherein

the identification unit identifies an individual piece of the tablet by using information for identifying an individual piece of the tablet.

(Supplementary Note 8)

A medication-taking management method comprising:

irradiating a tablet moving in a housing with light;

capturing an image of the irradiated tablet;

identifying the captured tablet, based on a feature of a tablet surface acquired from a captured image; and

outputting an image capture time indicating a time of capturing the captured image and an identification result associated with the image capture time.

(Supplementary Note 9)

The medication-taking management method according to supplementary note 8, further comprising

identifying the tablet, based on information representing a change of brightness in an area of the tablet in the captured image.

(Supplementary Note 10)

A management device comprising:

identification unit configured to identify a tablet subjected to image capturing, based on information representing a feature of a tablet surface acquired from a captured image acquired by capturing an image of a tablet irradiated with light; and

output control unit configured to output an image capture time indicating a time of acquiring the captured image and an identification result in association with each other.

(Supplementary Note 11)

The management device according to supplementary note 10, wherein

the identification unit identifies the tablet, based on information representing a change of brightness in an image of an area of the tablet in the captured image.

(Supplementary Note 12)

A management method comprising:

identifying a tablet subjected to image capturing, based on information representing a feature of a tablet surface acquired from a captured image acquired by capturing an image of a tablet irradiated with light; and

outputting an image capture time indicating a time of acquiring the captured image and an identification result in association with each other.

(Supplementary Note 13)

The management method according to supplementary note 12, further comprising

identifying the tablet, based on information representing a change of brightness in an image of an area of the tablet in the captured image.

(Supplementary Note 14)

A program storage medium storing a computer program that causes a computer to execute the processes of:

identifying a tablet subjected to image capturing, based on a feature of a tablet surface acquired from a captured image acquired by capturing an image of a tablet irradiated with light; and

outputting an image capture time indicating a time of capturing the captured image and an identification result in association with each other.

(Supplementary Note 15)

The program storage medium according to supplementary note 14, wherein

processing of the identification identifies the tablet, based on information representing a change of brightness in an image of an area of the tablet in the captured image.

(Supplementary Note 16)

A tablet-image capture device comprising:

irradiation unit configured to irradiate a tablet moving in a housing with light; and

image capture unit configured to acquire a captured image by capturing an image of the tablet irradiated with the light, the tablet being identified based on a feature of a tablet surface, wherein

the captured image is associated with an image capture time indicating a time of capturing the captured image, the image capture time being output in association with an identification result.

(Supplementary Note 17)

The tablet-image capture device according to supplementary note 16, wherein

the irradiation unit emits light where an irradiation area of light with respect to a first face and an irradiation area of light on a second face parallel to the first face fall within a predetermined range.

(Supplementary Note 18)

The tablet-image capture device according to supplementary note 16 or 17, further comprising:

detection unit configured to detect a signal based on input from an outside; and

control unit configured to control at least one of the irradiation means and the image capture means according to a detection result by the detection means.

While the invention has been particularly shown and described with reference to exemplary example embodiments thereof, the invention is not limited to these example embodiments. It will be understood by those of ordinary skill in the art that various changes in form and details may be made therein without departing from the spirit and scope of the present invention as defined by the claims.

This application is based upon and claims the benefit of priority from Japanese patent application No. 2017-126227, filed on Jun. 28, 2017, the disclosure of which is incorporated herein in its entirety by reference.

### REFERENCE SIGNS LIST

1 Tablet removal case

9 Tablet

10 Tablet removal management system

20 Tablet removal management system

30 Tablet removal management system

31 Irradiation unit

32 Image capture unit

33 Identification unit

34 Output control unit

100 Tablet-image capture device

101 Management device

102 Output device

110 Irradiation unit

120 Image capture unit

130 Identification unit

140 Recording unit

150 Output control unit

160 Control unit

200 Tablet-image capture device

201 Management device

230 Identification unit

240 Recording unit

260 Control unit

270 Detection unit

1. A medication-taking management system comprising:
light source configured to irradiate a tablet moving in a housing with light;
an image sensor configured to capture an image of the irradiated tablet; and
at least one processor configured to identify the captured tablet, based on a feature of a tablet surface acquired from a captured image acquired by the image sensor and
output an image capture time indicating a time of capturing the captured image and an identification result associated with the image capture time.

2. The medication-taking management system according to claim 1, wherein
the at least one processor identifies the tablet, based on a change of brightness in an area of the tablet in the captured image.

3. The medication-taking management system according to claim 2, wherein
the at least one processor identifies the tablet by using at least any one of a size, a shape, or a color of the tablet, or a character printed on the tablet.

4. The medication-taking management system according to claim 1, wherein
the light source emits light where an irradiation area of light with respect to a first face and an irradiation area of light on a second face parallel to the first face fall within a predetermined range.

5. The medication-taking management system according to claim 1, wherein
the at least one processor detects a signal based on input from an outside and
controls at least one of the light source and the image capture sensor according to a detection result.

6. The medication-taking management system according to claim 5, wherein
the at least one processor controls at least one of the light source and the image sensor according to information relating to the tablet.

7. The medication-taking management system according to claim 1, wherein
the at least one processor identifies an individual piece of the tablet by using information for identifying an individual piece of the tablet.

8. A medication-taking management method comprising:
irradiating a tablet moving in a housing with light;
capturing an image of the irradiated tablet;
identifying the captured tablet, based on a feature of a tablet surface acquired from a captured image; and
outputting an image capture time indicating a time of capturing the captured image and an identification result associated with the image capture time.

9. The medication-taking management method according to claim 8, further comprising
identifying the tablet, based on information representing a change of brightness in an area of the tablet in the captured image.

10-13. (canceled)

14. A non-transitory program storage medium storing a computer program that causes a computer to execute the processes of:
identifying a tablet subjected to image capturing, based on a feature of a tablet surface acquired from a captured image acquired by capturing an image of a tablet irradiated with light; and
outputting an image capture time indicating a time of capturing the captured image and an identification result associated with the image capture time.

15. The non-transitory program storage medium according to claim 14, wherein
processing of the identification identifies the tablet, based on information representing a change of brightness in an image of an area of the tablet in the captured image.

16-18. (canceled)

19. The medication-taking management system according to claim 5, wherein
the at least one processor controls the light source to start light irradiation, when detecting the tablet passing a separator of two spaces.

20. The medication-taking management system according to claim 5, wherein
the at least one processor controls the image sensor to capture the tablet located within an angle of view of the image sensor.
